(19) United States
(12) Patent Application Publication
(10) Pub. No.: US 2012/0204894 A1
(43) Pub. Date: Aug. 16, 2012

(54) HAIR GROWTH CONDITIONER

(76) Inventor: Valerie Maria Odoms, Gwynnoak, MD (US)

(21) Appl. No.: 13/371,360

(22) Filed: Feb. 10, 2012

Related U.S. Application Data
(60) Provisional application No. 61/441,421, filed on Feb. 10, 2011.

(51) Int. Cl. A61K 8/97; A61Q 7/00; A61Q 5/12

(52) U.S. Cl. 132/202; 424/727

(57) ABSTRACT

A solution to be applied topically to the human scalp in order to encourage hair growth, well-conditioned hair, control of dandruff, and overall health of the scalp. The present invention accomplishes these goals through a mixture of ingredients precisely proportioned to achieve a unique chemical composition that brings about maximum results. The ingredients in the solution include shea butter, extra virgin coconut oil, extra virgin olive oil, jojoba oil, brahmi and amla oil, castor oil, sweet almond oil, argan oil, sesame oil, vitamin E (tocopherol), vegetable glycerin, peppermint, rosemary, tea tree, and lavender oil.

### CONTINUITY

This is a non-provisional patent application of provisional patent application No. 61/441,421 filed on Feb. 10, 2011 and priority is claimed thereto.

### FIELD OF THE PRESENT INVENTION

The present invention relates to a solution to be applied topically to the human scalp in order to encourage hair growth, well-conditioned hair, control of dandruff, and overall health of the scalp. The present invention accomplishes these goals through a mixture of ingredients precisely proportioned to achieve a unique chemical composition that brings about maximum results. The ingredients in the solution include shea butter, extra virgin coconut oil, extra virgin olive oil, jojoba oil, brahmi and amla oil, castor oil, sweet almond oil, argan oil, sesame oil, vitamin E (tocopherol), vegetable glycerin, peppermint, rosemary, tea tree, and lavender oil.

### BACKGROUND OF THE PRESENT INVENTION

Topical solutions for hair growth and overall scalp health are, and always will be, in high demand. Hair loss afflicts huge numbers of men and women, and inspires in its sufferers an ardent desire to solve the problem. This desire can be seen by the large number of inventions that have been created to stop hair loss. There have truly been a myriad of patented products to promote scalp health and generate the growth of hair, yet, as in any field of application, there is always room for improvement. This is where the present invention comes in.

The present invention satisfies the aforementioned needs through its distinctive chemical composition. Well known, easily obtainable elements are ingeniously combined in a carefully calibrated mixture to bring about maximum results. These elements, such as shea butter, no less than nine different types of oils, as well as other substances, are proportionately fashioned in order to create the present invention.

Numerous topical solutions that promote hair growth and scalp health have received patents throughout the years, and it would be impossible to list references to all or even most of them. However, most patented solutions are comprised of a distinct mix of conventional ingredients combined in a uniquely proportionate manner. The present invention builds upon this legacy with an ingenious recipe of chemical composition for effective promotion of hair growth and overall scalp health.

U.S. Publication No. 2002/0168326 for Hair Conditioner and Scalp Treatment Solution by Lore, published on Nov. 14, 2002, shows a hair conditioner and scalp treatment solution. Lore's invention is a hair conditioner and scalp stimulator solution made of liquid extracts from coconut, lemon and lime fruit. The present invention, unlike Lore, does not utilize coconut, lemon or lime fruit.

U.S. Pat. No. 5,827,510 issued to Mesquitta on Oct. 27, 1998 is for a hair growth preparation to promote the growth of new hair and preserve existing hair. Mesquitta uses a combination of 10% to 40% of water, 1% to 10% of castor oil, and 50% to 90% of glycerin. Although the present invention also utilizes castor oil and vegetable glycerin, it does so in much smaller proportions than Mesquitta while using numerous other ingredients not used by Mesquitta.

U.S. Pat. No. 6,537,564 issued to Mabratu on Mar. 25, 2003 is for an all natural hair relaxer and conditioner. Mabratu uses natural components such as coffee, walnut oil, fish oil, carnauba milk, and honey mixed in predetermined proportions to provide hair straightening. Although the present invention also conditions hair, it employs ingredients far different than Mabratu.

U.S. Publication No. 2005/0053564 for Plant Extract to Enhance Hair Growth and Hair Restoration for Damaged Hair by Lieberman, published on Mar. 12, 2005, shows a topical solution designed to prevent or treat hair loss through the use of a compound made from shea butter, papaya and polysaccharides, preferably administered topically in the form of a lotion, gel, cream, or other suspension. Although the present invention, like Lieberman, utilizes shea butter and is applied topically, the present invention employs numerous other ingredients not used by Lieberman.

U.S. Pat. No. 7,166,300 issued to Dascalu on Jan. 23, 2007 is for an agent for inducing hair growth containing extracts of saw palmetto and swertia. Dascalu's agent can be ingested in a tablet or capsule, and can be used for both humans and non-human animals. The present invention, however, is a topical solution for humans only, and does not employ palmetto or swertia as ingredients.

### SUMMARY OF THE PRESENT INVENTION

The present invention is a hair growth conditioner composed of a variety of substances mixed according to a strict proportionate formula. When these substances are combined in the proportions specified in the present invention, the resultant unique chemical composition confers upon the user numerous benefits relating to hair growth and the health of the user's scalp.

Some of the benefits of the present invention, besides the stimulation of hair growth, are moisturizing and conditioning the user's hair, controlling dandruff and itchy scalp, treating psoriasis, repairing damaged hair, controlling frizzy hair, giving hair a smooth look and feel, minimizing hair shedding, strengthening and thickening the hair, making hair softer and more manageable, building hair body, and restoring luster and shine to the hair. The solution is non-greasy and for all types of hair.

The present invention is meant to be applied daily or every other day, after shampooing, on problem areas of the scalp/body. When used as a preconditioner, it should be left on hair or problem areas for at least 20 minutes, or even overnight. It can also be used as a preconditioner. For maximum effect, the user should apply heat to the problem areas in order that the oils within the present invention will be encouraged to go into the hair shaft.

### DETAILED DESCRIPTION OF THE PRESENT INVENTION

The present invention is a hair growth conditioner composed of a variety of substances mixed according to a strict proportionate formula. When these substances are combined in the proportions specified in the present invention, the resultant unique chemical composition confers upon the user numerous benefits relating to hair growth and the health of the user's scalp.

Table A shown below shows the percentage of each ingredient found within any quantity of the present invention.

TABLE A

| INGREDIENT | PCT. OF TOTAL |
|---|---|
| Shea butter | 42.6 |
| Extra virgin coconut oil | 10.7 |
| Extra virgin olive oil | 10.7 |
| Jojoba oil | 10.7 |
| Brahmi and amla oil | 5.3 |
| Castor oil | 5.3 |
| Sweet almond oil | 5.3 |
| Vegetable glycerin | 5.3 |
| Argan oil | 1.7 |
| Sesame oil | 1.7 |
| Vitamin E (tocopherol) | 0.67 |
| Peppermint | 0.043 |
| Rosemary | 0.036 |
| Tea tree | 0.014 |
| Lavender oil | 0.014 |

In the preferred embodiment of the present invention, the hair growth conditioner will be sold in a quantity of approximately 40 ounces. In order to make this quantity of the present invention, the following substances should be mixed in the proportions shown below in Table B:

TABLE B

| INGREDIENT | QUANTITY | |
|---|---|---|
| Shea butter | 16 | ounces |
| Extra virgin coconut oil | 4 | ounces |
| Extra virgin olive oil | 4 | ounces |
| Jojoba oil | 4 | ounces |
| Brahmi and amla oil | 2 | ounces |
| Castor oil | 2 | ounces |
| Sweet almond oil | 2 | ounces |
| Vegetable glycerin | 2 | ounces |
| Argan oil | 1.25 | ounces |
| Sesame oil | 1.7 | ounces |
| Vitamin E (tocopherol) | 0.67 | ounces |
| Peppermint | 12 | drops |
| Rosemary | 10 | drops |
| Tea tree | 4 | drops |
| Lavender oil | 4 | drops |

Below are brief explanations of the ingredients found in the present invention.

Shea butter is exceptionally rich and contains fatty acids that stimulate the skin's natural renewal processes and replenish lost oils to the scalp and hair, leaving it healthy and shiny. Because it is rich in Vitamins A and E, shea butter soothes dryness, repairs breakage and mends split ends. Shea butter is absorbed quickly and completely into the scalp without clogging pores, without leaving a greasy residue, and without causing build-up of oil or dandruff. Shea butter also helps heal a variety of scalp conditions, including dry scalp, psoriasis, eczema and dermatitis. It is rich in moisture and can shield against harmful ultraviolet radiation, protecting the hair from sun damage.

Extra virgin coconut oil is a light and penetrating oil that is cold-processed from fresh coconut milk. Extra virgin coconut oil is non-greasy, high in Vitamin E, and highly valued for its conditioning properties. It hydrates the scalp and produces shiny hair.

Extra virgin olive oil is moisturizing and very mild, making it suitable for children and those with sensitive skin. Extra virgin olive oil is high in oleic acid, which helps with skin cell regeneration and attracts moisture to the hair and skin.

Jojoba oil is a liquid wax ester derived from a desert plant that grows in the southwestern United States. It closely resembles the esters produced by the skin (known as sebum). This gives jojoba oil a natural affinity with the skin, making it readily absorbable without leaving a greasy or tacky residue on the skin. Jojoba oil is also excellent for hair and scalp conditioning. It has anti-inflammatory and anti-bacterial properties, as well as antioxidant properties that prevent it from turning rancid like other oils.

Brahmi and amla oil combine to bring about a unique building of body for the hair. With this combination, the amla ingredients act to condition and nourish the hair, while the Brahmi ingredients help to strengthen and add body to the hair.

Castor oil is known as a soothing and lubricating oil that is rich in fatty acids and acts as a humectant by attracting moisture to the hair and skin. Castor oil is superb for its conditioning and moisturizing properties, helping the ends of a user's hair become shiny and hiding the appearance of frizzy, damaged, and split ends. It reduces and prevents damage, leading to thicker and fuller hair for the user.

Sweet almond oil is a nutrient for skin conditioning with glucosides, vitamins, and minerals, and aids with eczema and itchy, dry, and inflamed skin. Sweet almond oil nourishes hair and smoothes hair cuticles, making the user's hair longer and thicker, adding shine to the hair, and controlling hair fall.

Vegetable glycerin is a humectant that holds moisture to the hair and skin. It has soothing properties that leaves the hair and skin smooth and silky.

Argan oil is loaded with essential fatty acids like linoleic acid, which is well known for its moisturizing and antioxidant properties. Argan oil can help a user avoid split ends and restore damaged hair. Other benefits of argan oil health include adding luster to dull hair, improving elasticity, and reducing hair loss due to breakage.

Sesame oil is a highly nourishing oil with many lubricating and healing properties. Sesame oil is known as a natural healing oil, as it is full of anti-bacterial properties. It helps clear scalp infection, especially of the fungal variety. Sesame oil strengthens the hair shaft and roots by nourishing them, and also promotes new hair growth.

Vitamin E (tocopherol) is an anti-oxidizing agent that helps to stabilize oils and prevent rancidity. Vitamin E (tocopherol) also has anti-oxidizing properties for the skin, promotes healing, and improves circulation within the scalp.

Peppermint helps to stimulate blood flow to the root of the hair. This helps the hair to receive proper nourishment and to grow correctly. Peppermint makes the scalp tingle, and this stimulates blood flow to the hair.

Rosemary is an essential oil that alleviates problems associated with dry hair, dandruff and itchy scalp.

Tea tree is an essential oil that works to purify the scalp and alleviate problems associated with scalp psoriasis and scalp dandruff.

Lavender oil is intended to soothe the scalp and calm the hair.

The brahmi and amla oil, castor oil, argan oil, and sesame oil are specifically agents for stimulating hair growth. The present invention, however, does more than stimulate hair growth. It also moisturizes and conditions the user's hair, controls dandruff and itchy scalp, treats psoriasis, repairs damaged hair, controls frizzy hair, gives hair a smooth look and feel, minimizes hair shedding, strengthens and thickens the hair, makes hair softer and more manageable, builds hair body, and restores luster and shine to the hair. The solution is non-greasy and is envisioned to be used on all types of hair. The result of the preferred embodiment of the present invention is a firm, butter-like cream that is preferably stored in a jar.

The present invention is meant to be applied daily or every other day, after shampooing, on problem areas of the scalp/body. When used as a preconditioner, it should be left on hair or problem areas for at least 20 minutes, or even overnight. It can also be used as a preconditioner. For maximum effect, the user should apply heat to the problem areas in order that the oils within the present invention will be encouraged to go into the hair shaft.

In the event that the present invention is additionally employed as a preconditioner, the present invention is envisioned to be applied sparingly to the scalp and throughout the ends of the hair as needed. In this embodiment, the present invention is employed as a leave-in conditioner, which is preferably employed once a week. The present invention is preferably applied to problematic areas daily, or every other day.

The present invention may also be applied to normal and/or dry hair as a deep conditioner. First, prior to shampooing the hair, apply the present invention to the scalp and throughout the hair to the ends. Next, cover the scalp with a plastic cap and sit under a hooded hairdryer, preferably set on medium setting, for approximately 20 to 30 minutes. It is via this method that the hair is deep conditioned. Next, simply shampoo the hair, then detangle the hair with a light conditioner for preferably one to two minutes. Finally, rinse and style the hair.

In the event that the present invention is to be applied to dry or damaged hair, the following method of use is preferred: First, prior to shampooing the hair, apply the present invention to the scalp and throughout the hair to the ends. Next, cover the scalp with a plastic cap and wrap the scalp with a scarf. The hair may be left in this position overnight if an intense conditioning is needed. Next, shampoo the hair, and then detangle the hair with a light conditioner for preferably one to two minutes. Finally, rinse and style the hair as needed.

It is envisioned that the present invention is not limited as to the duration of use on hair. It is encouraged, however, to be applied to hair consistently in order to achieve the best results, and maintain healthy strong hair.

I claim:

1) A hair growth conditioner for hair comprising:
a shea butter;
an extra virgin coconut oil;
an extra virgin olive oil;
a jojoba oil;
a Brahmi oil;
an amla oil;
a castor oil;
a sweet almond oil;
a vegetable glycerin;
an argan oil;
a sesame oil;
a vitamin E;
a peppermint;
a rosemary;
a tea tree; and,
a lavender oil.

2) The hair growth conditioner for hair of claim 1, wherein said shea butter, said extra virgin coconut oil, said extra virgin olive oil, said jojoba oil, said brahmi oil, said amla oil, said castor oil, said sweet almond oil, said vegetable glycerin, said argan oil, said sesame oil, said vitamin E, said peppermint, said rosemary, said tea tree, and said lavender oil are evenly distributed.

3) A method for applying hair growth conditioner, comprising:
applying a mixture of a shea butter, extra virgin coconut oil, an extra virgin olive oil, a jojoba oil, a Brahmi oil, an amla oil, a castor oil, a sweet almond oil, a vegetable glycerin, an argan oil, a sesame oil, a vitamin E, a peppermint, a rosemary, a tea tree and, a lavender oil to the scalp;
covering the scalp with a plastic cap;
placing the scalp under a hooded hairdryer;
setting the hooded hairdryer on medium setting for at least 20 minutes;
washing the hair with a shampoo;
rinsing the hair;
applying a light conditioner to the hair for at least one minute; and
rinsing the hair.

4) The method of claim 3, wherein said mixture of shea butter, extra virgin coconut oil, extra virgin olive oil, jojoba oil, Brahmi oil, amla oil, castor oil, sweet almond oil, vegetable glycerin, argan oil, sesame oil, vitamin E, peppermint, rosemary, tea tree and, lavender oil are evenly distributed.

5) A hair growth conditioner consisting of:
a shea butter;
an extra virgin coconut oil;
an extra virgin olive oil;
a jojoba oil;
a Brahmi oil;
an amla oil;
a castor oil;
a sweet almond oil;
a vegetable glycerin;
an argan oil;
a sesame oil;
a vitamin E;
a peppermint;
a rosemary;
a tea tree; and,
a lavender oil.
